(19) United States
(12) Patent Application Publication
(10) Pub. No.: US 2020/0337578 A1
(43) Pub. Date: Oct. 29, 2020

(54) WEARABLE ECG AND AUSCULTATION MONITORING SYSTEM WITH SOS AND REMOTE MONITORING

(71) Applicant: ROHINI SHANKAR, HYDERABAD (IN)

(72) Inventor: ROHINI SHANKAR, HYDERABAD (IN)

(21) Appl. No.: 16/961,936

(22) PCT Filed: Jan. 9, 2019
(86) PCT No.: PCT/IB2019/050144

§ 371 Date: Jul. 14, 2020

(30) Foreign Application Priority Data
Jan. 9, 2018 (IN) 201841001028

(51) Int. Cl. A61B 5/0402; A61B 5/0205; A61B 5/11; A61B 5/00; A61B 5/1455

(52) U.S. Cl. CPC A61B 5/0402; A61B 5/0205; A61B 5/1102; A61B 5/1112; A61B 5/0022; A61B 5/4866; A61B 5/681; A61B 5/14551; A61B 5/747; A61B 5/1118

(57) ABSTRACT

Wearable ECG and auscultation monitoring system with SOS and remote monitoring. A wearable device for monitoring cardiac parameters of a user is provided. The device comprises a first electrode to measure electrical potential on a first portion of the user's body, a second electrode to measure electrical potential on a second portion of the user's body and a processor to measure the potential difference between the first electrode and the second electrode. It further comprises a sensor for capturing Seismocardiography, Phonocardiogramand Ballistocardiogram generated by the motion of the heart wall and the blood flow, display module for displaying the generated information, wireless communication modules for communicating and transferring the information generated by the device to a smart phone application of the user and to a remote data processing system.

### FIELD OF THE INVENTION

The subject matter in general relates to a device that has the capability of capturing and recording different cardiac parameters of a user. More particularly, but not exclusively, the subject matter relates to a wearable electrical device for diagnosing cardiac related diseases at early stages, which may additionally be used for remote diagnostics of the patients.

### BACKGROUND OF THE INVENTION

Unless otherwise indicated herein, the materials described in this section are not prior art to the claims in this application and are not admitted to being prior art by inclusion in this section.

### DISCUSSION OF THE RELATED ART

Fitness and health has an immense co-relation to the functioning of the heart. Proper understanding of information from the heart in the form of various cardiac parameters is an important tool to improve one's fitness and lifestyle. Studies signifies that higher heart rate reduces the quality of life as well as increases the chances of heart failure, myocardial infarction and other such cardiac disorders. Hence monitoring of cardiac parameters has become significantly important for maintaining a healthy and balanced life.

Conventionally, Heart Rate is typically computed using different diagnostic equipment such as Electrocardiogram, pressure sensors near arteries or veins or by the use of pulse oximeters. Moreover, mechanical parameters of the heart including sounds and murmurs made by the heart during a cardiac cycle is typically captured by an acoustic medical device called stethoscope. This requires a Doctor/Physician to have good listening skills and can only be taken in a calm environment.

So, in conventional diagnostic systems, a medical expert is required to diagnose and study various cardiac parameters at regular intervals. The details of various cardiac parameters cannot be monitored at regular intervals of time using conventional systems. Sometimes, it might be necessary to monitor the health of the user by a medical practitioner from a distant place. Moreover, it might be necessary for the patient to communicate to others in case of extreme emergency.

In light of the foregoing discussion, there is a need for an improved system for diagnosing and monitoring the cardiac parameters of a patient at regular intervals with out the involvement of a medical expert.

### SUMMARY OF THE INVENTION

In one aspect, a wearable device is provided for diagnosing and monitoring the cardiac parameters of a user. The device comprises a first electrode to measure electrical potential on a first portion of the user's body, a second electrode to measure electrical potential on a second portion of the user's body and a processor to measure the potential difference between the first electrode and the second electrode. The device further comprises a sensor for capturing different cardiac parameters generated by the motion of the heart wall and the blood flow, a display module for displaying the information generated from detecting and recording the cardiac parameters of the user, a first wireless communication module for communicating and transferring the information to a smart phone application and a second wireless communication module for communicating and transferring the information to a remote data processing system. The device detects and records various cardiac parameters of the user such as Electrocardiogram, Seismocardiogram, Phonocardiogram, Ballistocardiogram, Blood Pressure, Heart Rate, Blood Oxygen saturation levels, Respiratory Rate etc.

### BRIEF DESCRIPTION OF THE DRAWINGS

FIG. 1 illustrates a wearable device that can be worn on the left-hand wrist of the user.

FIG. 2 illustrates the Einthoven triangle formed from the typical electrode placement locations on the user's body.

FIG. 3 illustrates a wearable device that can be worn on the left-hand wrist of the user in Lead I configuration with positive electrode (under the watch) touching the left-hand wrist and right index finger touching the negative electrode on the side of the watch.

FIGS. 4a and 4b illustrates the typical electrocardiographic lead configuration locations on the device according to the examples of the disclosure.

FIG. 5 illustrates a user interactive touch screen Liquid crystal display with touch screen feature to interact with the watch. It also illustrates the location of typical pedometer in the watch.

FIG. 6 illustrates a typical EKG signal.

FIG. 7 illustrates the ideal placement of the wearable device or watch on the chest of the user for capturing auscultations of heart and lungs.

FIG. 8 illustrates typical location of the piezoelectric sensor or MEMS accelerometer for capturing Seismocardiography (SCG), Phonocardiogram (PCG) and Ballistocardiogram generated by the motion of the heart wall and blood flow.

FIG. 9 illustrates a typical PCG signal.

FIG. 10 illustrates an ECG taken in Lead II configuration.

FIG. 11 illustrates ECG taken in Lead III configuration.

FIG. 12 illustrates a typical PPG graphical representation.

### DETAILED DESCRIPTION OF THE INVENTION

The following detailed description includes references to the accompanying drawings, which form part of the detailed description. The drawings show illustrations in accordance with example embodiments. These example embodiments are described in enough details to enable those skilled in the art to practice the present subject matter. However, it may be apparent to one with ordinary skill in the art that the present invention may be practised without these specific details. In other instances, well-known methods, procedures and components have not been described in detail so as not to unnecessarily obscure aspects of the embodiments. The embodiments can be combined, other embodiments can be utilized or structural and logical changes can be made without departing from the scope of the invention. The following detailed description is, therefore, not to be taken as a limiting sense.

In this document, the terms “a” or “an” are used, as is common in patent documents, to include one or more than one. In this document, the term “or” is used to refer to a non-exclusive “or”, such that “A or B” includes “A but not B”, “B but not A”, and “A and B”, unless otherwise indicated.

It should be understood, that the capabilities of the invention described in the present disclosure and elements shown in the figures may be implemented in various forms of hardware, firmware, software, recordable medium or combinations thereof.

### Overview

The present invention discloses a wearable electrical device 101 for diagnosing and monitoring cardiac related diseases of a user. The device comprises a first electrode 103 to measure electrical potential on a first portion of the user's body, a second electrode 104 to measure electrical potential on a second portion of the user's body and a processor to measure the potential difference between the first electrode 103 and the second electrode 104. It further comprises a sensor for capturing different cardiac parameters generated by the motion of the heart wall and the blood flow. A display module 102 is arranged for displaying the information generated from detecting and recording the cardiac parameters of the user. The device 101 also includes a first wireless communication module for communicating and transferring the information generated by the device 101 to a smart phone application of the user and a second wireless communication module for communicating and transferring the information generated by the device 101 to a remote data processing system and also for initiating SOS call and SMS feature. The device 101 detects and records various cardiac parameters of the user such as Electrocardiogram, Seismocardiogram, Phonocardiogram, Ballistocardiogram, Blood Pressure, Heart Rate, Blood Oxygen saturation levels, Respiratory Rate etc. The device 101 is configured such that an alert is generated either at the device 101, at the remote data processing system or at the smart phone application if the device 101 detects abnormalities in the cardiac parameters of the user. The device 101 comprises a pulse oximeter to monitor the blood oxygen saturation levels, a heart rate monitoring sensor to calculate the heart rate of the user, an in-built module 122 to detect the step count of the user and a module to monitor the physical activity of the user. An external power supply unit is removably engaged with the device 101 to charge the device 101 in addition to an in-built battery module. The device 101 is connected to the remote data processing system through wireless communication modules such as Wi-Fi and Bluetooth. Further, the device 101 comprises a GPS module which identifies the location of the device 101.

### Construction of Wearable Device

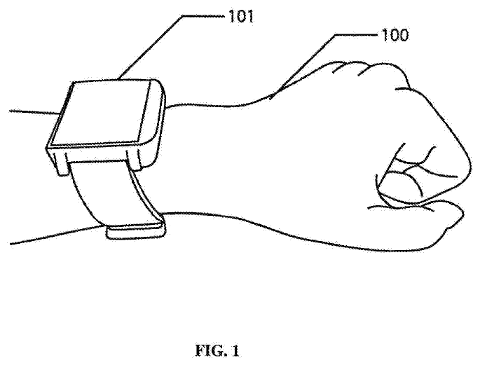

Referring to the figures, and more particularly to FIG. 1, a wearable device 101 for monitoring cardiac parameters of a user is disclosed. The device 101 may be fashioned into a wearable accessory that can be worn on the hands, wrists, legs, feet or any other part of the user's body. The device 101 may be designed in the form of a watch, ring, pendant, wrist-band, bracelet, etc.

In an embodiment, the device 101 may be affixed to a limb of the human body such as a wrist or ankle. The wearable device 101 can be worn on the left or right wrist/ankle. As illustrated in FIG. 1, the device 101 is wearable in the form of a watch, in accordance with an embodiment.

The device 101 detects and records the cardiac parameters of the user selected from the group comprising of Electrocardiogram, Seismocardiogram, Phonocardiogram, Ballistocardiogram, Blood Pressure, Heart Rate, Blood Oxygen saturation levels, Respiratory Rate, and combinations thereof.

The device 101 comprises a first electrode 103 to measure electrical potential on a first portion of the user's body, a second electrode 104 to measure electrical potential on a second portion of the user's body and a processor to measure the potential difference between the first electrode 103 and the second electrode 104.

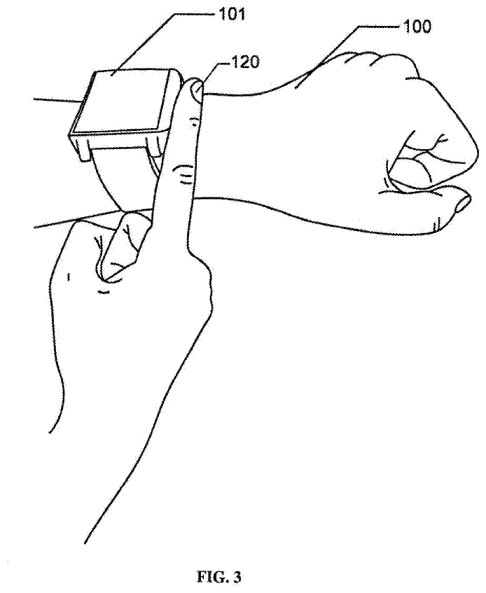

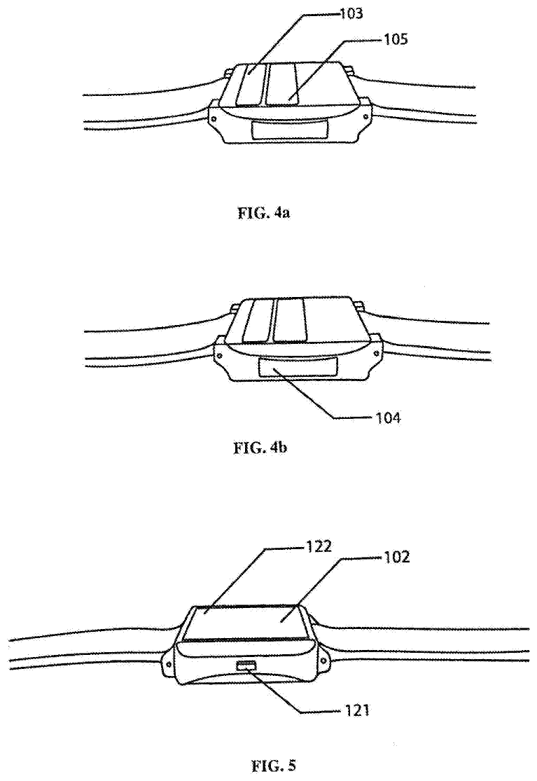

FIGS. 4a and 4b illustrates the arrangement of the first electrode 103 at the bottom portion of the device 101 and the second electrode 104 at the side portion of the device 101, in accordance with an embodiment. The first electrode 103 acts as a positive electrode and the second electrode 104 acts as a negative electrode. The potentials at the first electrode 103 and the second electrode 104 can be measured with respect to the ground.

As illustrated in FIG. 4a, the first electrode 103 and the ground electrode 105 can be positioned such that they are in direct contact with the wrist i.e., on the underside of the wearable device 101, while the second electrode 104 in FIG. 4b can be positioned such that it is not in direct contact with any portion of the user's body (as illustrated in FIG. 3).

FIG. 3 illustrates the usage of the device 101 in order to capture and record an electrocardiographic measurement, in accordance with an embodiment. The user may place a part of his or her body, such as a finger, on the second electrode 104 that is not in direct contact with the user's body.

In another embodiment, the user may place the second electrode 104 in contact with either ankle. Once the second electrode 104 comes into contact with a portion of the user's body, an electrocardiographic measurement that measures the potential difference between the portion of the body in contact with the first electrode 103 and the portion of the body in contact with the second electrode 104 may be captured and recorded.

FIG. 3 illustrates an exemplary placement of the device 101 on the user. The wearable device 101 is shown as being worn on the left wrist 100 of the user. FIG. 3 illustrates an example placement where the first electrode 103 can be on the underside of the wearable device 101, with the first electrode 103 touching the left wrist 100 of the user. In order to record an electrocardiographic measurement, the user may place one of the right-hand fingers 106 on the second electrode 104.

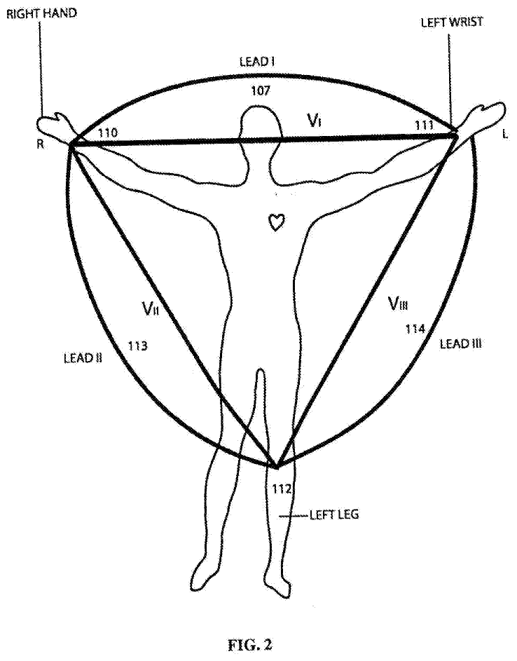

The device 101 measures the potential difference between the left wrist of the user and one of the right-hand fingers 106 of the user. An electrocardiographic measurement obtained in this manner can correspond to the Lead I configuration, as illustrated in FIG. 2, at side 107 of Einthoven's triangle. Since the positive electrode is placed at the left wrist 100, while the negative electrode is being touched by a right-hand finger 106, equation 1 can be used to characterize the electrocardiographic measurement.

FIG. 2 illustrates various electrocardiographic lead configurations in accordance with an embodiment. The electrocardiographic electrodes can be placed on the right wrist side (from the perspective of the patient) 110, the left wrist side 111, or on the left leg side 112. Placing the electrodes in this manner can form what is known in the art as Einthoven's triangle. Einthoven's triangle can have sides, 110, 111, and 112. Each side of the triangle can represent a lead configuration for taking an electrocardiographic measurement. For instance, side 107 of the triangle, known in the art as the Lead I configuration, can represent measuring the potential difference between the left wrist 100 and the right wrist 110. Side 113 of the triangle, known in the art as the Lead II configuration, can represent measuring the potential difference between the left leg and the right wrist 110. Side 114 of the triangle, known in the art as the Lead III configuration, can represent measuring the potential difference between the left leg and the left wrist. By measuring the potential difference between any two-electrode combination, an electrocardiographic measurement can be recorded. Each lead configuration can also have two possible ways to produce measurements. For instance, the Lead I configuration 107 can measure the potential difference between the left wrist (V_{L}) and the right wrist (V_{R}). This potential difference (V_{I}) can be expressed as the difference between the voltages measured at the right wrist and the left wrist, for example, as expressed in equation 1:

V_{I}=V_{L}−V_{R} (1)

The V_{L} and V_{R} potentials can be measured with respect to a ground electrode, for example, placed on the right leg or left wrist. The potential difference between the right wrist and the left wrist can also be expressed as:

−V_{I}=V_{R}−V_{L} (2)

Equations 1 and 2 thus can be inversions of one another. This can mean that depending on which electrode is the positive electrode (i.e., the number being subtracted from) and which electrode is the negative electrode (i.e., the number being subtracted), and also which electrode is placed on which hand, the results can be inverted with respect to one another.

The Lead II configuration 113 in FIG. 2 can measure the potential difference between the left leg and the right wrist. This potential difference (V_{II}) can be expressed as the difference between the voltages measured at the right wrist (V_{R}) and left ankle (V_{F}), for example, as expressed in equation 3:

V_{II}=V_{F}−V_{R} (3)

The potential difference between the right wrist and the left ankle can also be expressed as:

−V_{II}=V_{R}−V_{F} (4)

Equations 3 and 4 thus can be inversions of one another. This can mean that depending on which electrode is the positive electrode and which electrode is the negative electrode, the results can be inverted with respect to one another. This can be accounted for by configuring the wearable device to the user (left hand user or a right-hand user)

The Lead III configuration 114 in FIG. 2 can measure the potential difference between the left leg 112 and the left wrist 111. This potential difference (V_{III}) can be expressed as the difference between the voltages measured at the left ankle (V_{F}) and the left wrist (V_{L}), for example, as expressed in equation 5:

V_{III}=V_{F}−V_{L} (5)

−V_{III}=V_{L}−V_{F} (6)

Equations 5 and 6 thus can be inversions of one another. This can mean that depending on which electrodes is the positive electrode and which electrode is the negative electrode, the results can be inverted with respect to one another. This can be accounted for by configuring the wearable device 101 to the user (left hand user or a right-hand user).

Because of the fact that there can be lead inversions, medical practitioners often have to take care as to the placement of the positive electrode and the negative electrode to ensure accurate processing of an electrocardiographic measurement. In case of the wearable device 101 of the present invention, the device 101 should be configured for a particular user to ensure accuracy of the acquired readings.

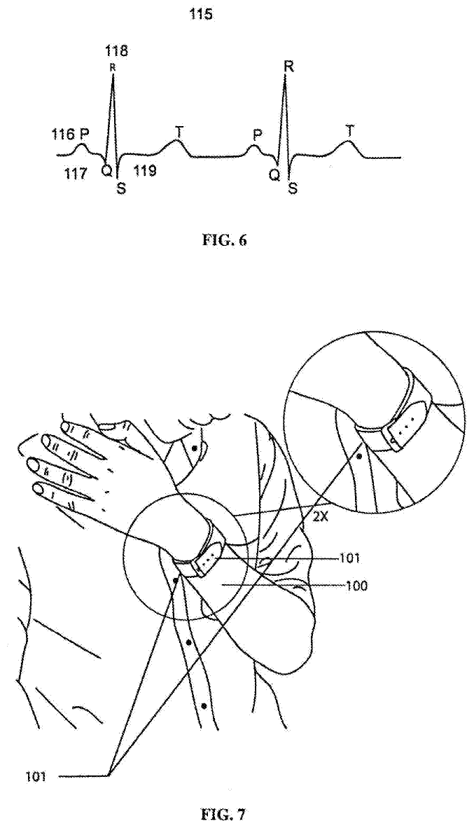

FIG. 6 illustrates an example electrocardiographic measurement 115 of the present invention, wherein the measurement 115 is made when the electrodes are placed in the Lead I configuration 107, with the positive electrode on the left wrist side and the negative electrode on the right wrist side.

FIG. 10 illustrates an example embodiment in which the electrocardiographic measurement is made when the electrodes are placed in the Lead II configuration 113 (of FIG. 2), with the positive electrode on the right wrist side 110 and the negative electrode on the left leg side 112.

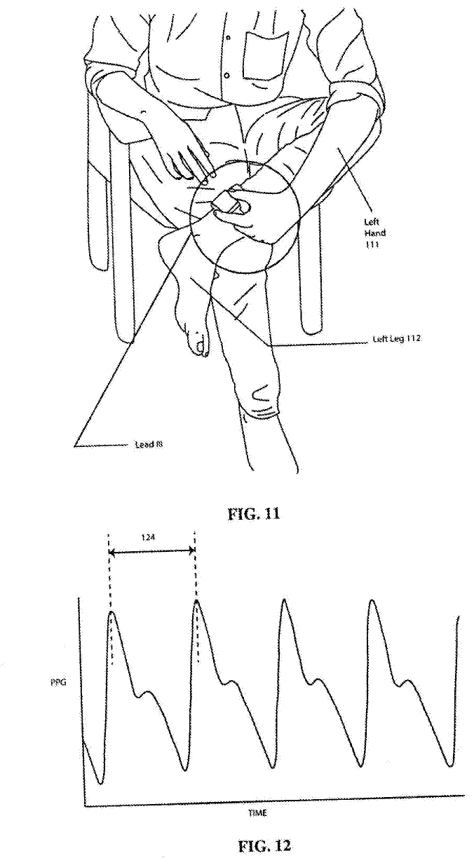

FIG. 11 illustrates another example embodiment, wherein, the electrocardiographic measurement is made when the electrodes are placed in the Lead I configuration 114 (of FIG. 2), with the positive electrode on the left wrist side 111 and the negative electrode on the left leg side 112.

Referring to FIG. 6, the potential difference between the two electrodes can vary based on electrical signals being produced by the heart as part of its normal function. Measurement 115 can represent the varying potential between the two electrodes over one cycle of a heartbeat. For instance, initially at 116, the potential difference can rise and then fall for a brief duration. This initial rise and fall can correspond to atrial depolarization of the heart muscle and is known in the art as a P-wave. At 117, the potential between the two electrodes can fall for a brief period of time. The fall can correspond to septal depolarization and is known in the art as a Q-wave. At 118, the potential difference can have a sharp rise. The rise can correspond to apical depolarization (i.e., when the majority of the ventricle tissue depolarizes) and is known in the art as an R-wave. At 119, the potential difference can fall. The fall can correspond to left ventricular depolarization and is known in the art as an S-wave.

FIG. 7 illustrates an exemplary heart and lung auscultation measurement device shown according to the examples of the present invention. In the example of FIG. 8 the wearable device 101 is shown as being worn on the left wrist 100 of the user 100 with bottom of the wearable device 101 is on the back side of the wrist. In the example of FIG. 7 wearable device 101 is placed on the anterior chest wall of the user. The wearable device 101 is equipped with the ability to measure precordial accelerations on patient's chest by means of a sensors typically piezoelectric sensor or MEMS accelerometer located just below the upper surface of the face of the watch for capturing Seismocardiography (SCG), Phonocardiogram (PCG) and Ballistocardiogram generated by the motion of the heart wall and blood flow and converting them into electrical signals which are sent to the signal processor for filtering using Higher order FIR band pass filter at some particular sampling frequency.

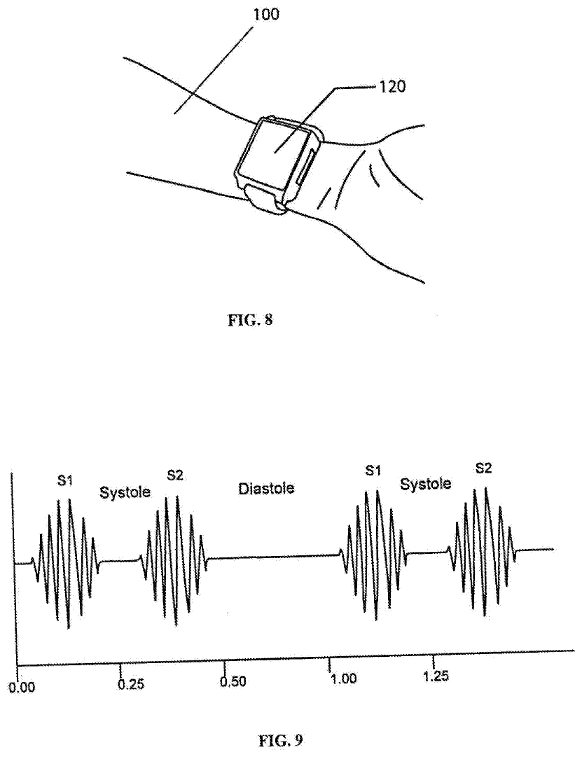

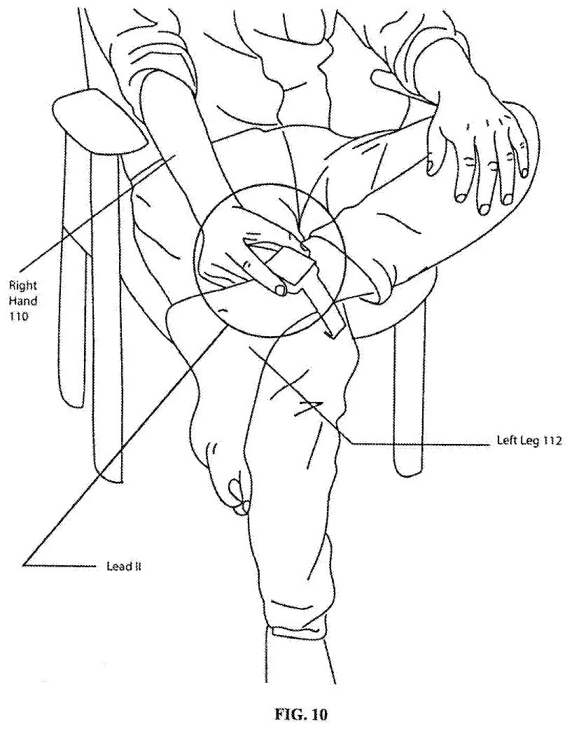

FIG. 9 illustrates an example Phonocardiogram (PCG) graphical representation of heart beat, wherein, S1 represents systolic heart beat and S2 represents diastolic heartbeat. In the illustrated graph for PCG, S1 and S2 represent one heartbeat cycle.

FIG. 12 illustrates a typical Photoplethysmography (PPG) graph. PPG is a non-invasive method that takes measurements at the surface of the skin with an optical technique used to detect volumetric changes in blood in peripheral circulation. The wearable device may be equipped with typical PPG sensors. With reference to FIG. 4a, 105 illustrates atypical location of the Green light PPG sensor for the watch used for optical heart rate monitoring (OHRM). The green light is projected on to the skin of the subject and the amount of light reflected back and the amount of light absorbed is captured using photodiodes. The date from the photodiode is used to plot PPG waveform. Using the wave form (in FIG. 12), peak to peak time interval 124 is used to calculate the continuous heart rate or number of heart of the user.

FIG. 5 illustrates a wearable device 101 that should have various methods to output the processed EKG, heart and lung auscultation signals. The output can be displayed on display type known in the art, for example, Liquid Crystal Displays (LCD), Light Emitting Diode Displays (LED), Cathode Ray Tube Displays (CRT), Orthogonal Liquid Crystal Displays (OLCD), Organic Light Emitting Diode Displays (OLED) or any other type of display currently existing or which may exist in the future. It is also possible to combine the video display with user interface by use of technologies such as touch screen or tapping or double tapping of display. The device which includes a touch screen large enough to incorporate all or a portion of the user interface for the device. Contrast and brightness capability can also be added to the display. It is also possible to put in the activity features that engages and inspires the users to be more fit and health conscious, by consciously and gradually altering their lifestyle.

The device further comprises a sensor for capturing Seismocardiography, Phonocardiogram and Ballistocardiogram generated by the motion of the heart wall and the blood flow.

The device comprises a pulse oximeter to monitor the blood oxygen saturation levels, wherein the remote data processing system is configured to receive information from the pulse oximeter.

The device comprises a heart rate monitoring sensor to calculate the heart rate of the user, wherein the remote data processing system is configured to receive information from the heart rate monitoring sensor. The heart rate of the user calculated by the device is used for calculating the respiration rate of the user.

The device may comprise an in-built module 122 to detect the step count of the user, wherein the data processing system is configured to receive information from the in-built module. In an embodiment, the in-built module 122 is selected from the group comprising of gyroscope, accelerometer, and combinations thereof.

The device 101 may further comprise a module to monitor the physical activity of the user, thereby measuring the number of calories spent by the user.

The device 101 comprises an in-built battery module, wherein the in-built battery module provides power for the functioning of the device. In an embodiment, the in-built battery may be replaceable or may be rechargeable, wherein the in-built battery is recharged using the external power supply unit. In an embodiment, the external power supply unit may be selected from the group comprising of unregulated, linear regulated, switching, ripple-regulated types and combinations thereof.

In an embodiment, the device 101 is configured to generate an alert if the device detects abnormalities in the cardiac parameters of the user. In another embodiment, the device 101 is configured to generate an alert at the remote data processing system, if the device detects any abnormalities in the recorded cardiac parameters of the user. In yet another embodiment, the device 101 is configured to generate an alert at the smart phone application of the user, if the device detects any abnormalities in the recorded cardiac parameters of the user. The device 101 may also be configured to generate an alert to keep a track on the medication of the user. In an embodiment, the alert may be one or more of alarm, vibration, blinker among others.

The data obtained from the different modules or sensors of the device may be communicated to the remote data processing system. The remote data processing system is configured to receive information from the second wireless communication module of the device, process the received information and display information based on the processing. The remote data processing system may be either a mobile device, laptop, computer, tablet, or server among others. The user operating the remote data processing system may monitor the information obtained as need be. In an embodiment, instructions and input to various modules of the device may be communicated from the data processing system to the device. The functioning of the device may also be controlled using the data processing system.

In an embodiment, the second wireless communication module is selected from the group comprising of Bluetooth, Wi-Fi, and combinations thereof.

A user login may be provided for users for controlling and monitoring the data. In an embodiment, a data log may be generated corresponding to the results obtained from Electrocardiogram, Seismocardiogram, Phonocardiogram, Ballistocardiogram, Blood Pressure, Heart Rate, Blood Oxygen saturation levels, Respiratory Rate etc. as per the user requirement.

In an embodiment, a GPS module may be used in detecting the location of the device and the user. The display of the device may be used to switch the GPS module on. As an example, the data processing system may be an application installed in the device of the user. The user may be able to locate the exact location of the device using the application.

The device also comprises an SOS call and SMS emergency feature. The device may have the ability to measure Electrocardiograph (EKG) and Phonocardiogram (PCG) at the same time and can also stream the data simultaneously to the LCD screen and on the remote server for remote monitoring. The wearable device may include computation learning algorithms to looks for abnormalities in the captured data and may notify the user if there is an abnormality by visual, aural and haptic feedback. In case of emergency, the wireless communication module can be activated by the computing device or by user interface that may include capacitive touch or by tapping/double tapping of device or by a tactile switch as illustrated in the FIG. 5. Upon initiating the wireless communication module, the device establishes communication with the preprogramed telephone numbers along with approximate location of the user. The user can communicate with the person on other side using the built-in microphone and loudspeaker.

The device assists to monitor the health condition of a patient from a distant place, wherein, various cardiac and other physical parameters of the user can be monitored. The device helps in increased attention on the health and physical activity of the user. Hence, it is observed that the proposed invention promotes the concept of virtual doctor.

It shall be noted that the processes described above are described as sequence of steps; this was done solely for the sake of illustration. Accordingly, it is contemplated that some steps may be added, some steps may be omitted, the order of the steps may be re-arranged, or some steps may be performed simultaneously.

Although embodiments have been described with reference to specific example embodiments, it will be evident that various modifications and changes may be made to these embodiments without departing from the broader spirit and scope of the system and method described herein. Accordingly, the specification is to be regarded in an illustrative rather than a restrictive sense.

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above includes many specifications; these should not be construed as limiting the scope of the invention but as merely providing illustrations of some of the personally preferred embodiments of this invention. Thus, the scope of the invention should be determined by the appended claims and their legal equivalents rather than by the examples given.

1. A wearable device for monitoring cardiac parameters of a user, the device comprising:
a) a first electrode, to measure electrical potential on a first portion of the user's body;
b) a second electrode, to measure electrical potential on a second portion of the user's body;
c) a processor, to measure the potential difference between the first electrode and the second electrode;
d) a sensor for capturing Seismocardiography, Phonocardiogram and Ballistocardiogram generated by the motion of the heart wall and the blood flow.
e) a display module, for displaying the information generated from detecting and recording the cardiac parameters of the user; and
f) a first wireless communication module, for communicating and transferring the information generated by the device to a smart phone application of the user; and
g) a second wireless communication module, for communicating and transferring the information generated by the device to a remote data processing system and also for initiating SOS call and SMS feature.

2. The device as claimed in claim 1, wherein the remote data processing system is configured to receive information from the second wireless communication module of the device, process the received information and display information based on the processing.

3. The device as claimed in claim 1, wherein the first electrode is located at the bottom portion of the device.

4. The device as claimed in claim 1, wherein the second electrode is located at the side portion of the device.

5. The device as claimed in claim 1, wherein the device detects and records the cardiac parameters of the user selected from the group comprising of Electrocardiogram, Seismocardiogram, Phonocardiogram, Ballistocardiogram, Blood Pressure, Heart Rate, Blood Oxygen saturation levels, Respiratory Rate, and combinations thereof.

6. The device as claimed in claim 1, wherein the device detects and records Electrocardiogram by placing electrodes in a manner forming Einthoven's Triangle, wherein each side can represent a lead configuration for taking electrocardiographic measurement.

7. The device as claimed in claim 1, wherein the device is configured to generate an alert if the device detects abnormalities in the cardiac parameters of the user.

8. The device as claimed in claim 1, wherein the device is configured to generate an alert at the remote data processing system, if the device detects any abnormalities in the recorded cardiac parameters of the user.

9. The device as claimed in claim 1, wherein the device is configured to generate an alert at the smart phone application of the user, if the device detects any abnormalities in the recorded cardiac parameters of the user.

10. The device as claimed in claim 1, wherein the device is configured to generate an alert to keep a track on the medication of the user.

11. The device as claimed in claim 1, the wireless communication module is selected from the group comprising of Bluetooth, Wi-Fi, GSM, GPRS, and combinations thereof.

12. The device as claimed in claim 1, wherein the device comprises a GPS module to identify location of the device and the user, wherein the remote data processing system is configured to receive the information from the GPS module.

13. The device as claimed in claim 1, wherein the device comprises a pulse oximeter to monitor the blood oxygen saturation, wherein the remote data processing system is configured to receive information from the pulse oximeter.

14. The device as claimed in claim 1, wherein the device comprises a heart rate monitoring sensor to calculate the heart rate of the user, wherein the remote data processing system is configured to receive information from the heart rate monitoring sensor.

15. The device as claimed in claim 14, wherein the heart rate of the user calculated by the device is used for calculating the respiration rate of the user.

16. The device as claimed in claim 1, wherein the device comprises an in-built module to detect the step count of the user, wherein the data processing system is configured to receive information from the in-built module.

17. The device as claimed in claim 16, wherein the in-built module is selected from the group comprising of gyroscope, accelerometer, and combinations thereof.

18. The device as claimed in claim 1, wherein the device comprises a module to monitor the physical activity of the user, thereby measuring the number of calories spent by the user.

19. The device as claimed in claim 1, wherein the device comprises an in-built battery module, wherein the in-built battery module provides power for the functioning of the device.

20. The device as claimed in claim 1, wherein the device comprises an external power supply unit, wherein the external power supply unit is configured to charge the device and be removably engaged with the device.

21. The device as claimed in claim 1, wherein smart phone application of the user is configured to communicate instructions to different modules of the device.

22. The device as claimed in claim 1, wherein the remote data processing system is configured to communicate instructions to different modules of the device.
